(19) United States
(12) Patent Application Publication
(10) Pub. No.: US 2020/0163649 A1
(43) Pub. Date: May 28, 2020

(54) Estimating Phase Velocity Dispersion in Ultrasound Elastography Using a Multiple Signal Classification

(71) Applicant: Mayo Foundation for Medical Education and Research, Rochester, MN (US)

(72) Inventors: Matthew W. Urban, Rochester, MN (US); Piotr Kijanka, Krakow (PL)

(21) Appl. No.: 16/619,502

(22) PCT Filed: Jun. 5, 2018
(86) PCT No.: PCT/US2018/036050

§ 371 Date: Dec. 5, 2019

Related U.S. Application Data
(60) Provisional application No. 62/515,345, filed on Jun. 5, 2017.

(51) Int. Cl. A61B 8/08

(52) U.S. Cl. CPC A61B 8/485; A61B 8/5269; A61B 8/08; A61B 8/5223

(57) ABSTRACT

Methods for estimating the phase velocity of a shear wave from ultrasound data are described. An ultrasound system is used to measure one or more shear waves propagating in a region-of-interest in a subject, and from this data the phase velocity of the one or more shear waves can be estimated. In general, the phase velocity is estimated from a wavenumber spectrum computed from temporal frequency data generated by Fourier transforming the ultrasound data along one or more temporal dimensions. A multiple signal classification (i.e., MUSIC) technique is adapted to separate the temporal frequency data into signal and noise subspaces, from which wavenumber spectra can be estimated based, at least in part, on an estimation function.

### CROSS-REFERENCE TO RELATED APPLICATIONS

This application claims the benefit of U.S. Provisional Patent Application Ser. No. 62/515,345, filed on Jun. 5, 2017, and entitled “ESTIMATING PHASE VELOCITY DISPERSION IN ULTRASOUND ELASTOGRAPHY USING A MULTIPLE SIGNAL CLASSIFICATION,” which is herein incorporated by reference in its entirety.

### STATEMENT REGARDING FEDERALLY SPONSORED RESEARCH

This invention was made with government support under DK092255 and HHSN268201500021C awarded by the National Institutes of Health. The government has certain rights in the invention.

### BACKGROUND

Shear wave elastography (“SWE”) is a method that is used to make noninvasive, quantitative measurements of various mechanical properties. In this technique, focused ultrasound beams are used to “push” the tissue, which generates a propagating shear wave. The velocity of the waves is modified by the mechanical properties of the tissue. In most SWE applications, the medium is assumed to be elastic, homogeneous, isotropic, linear, and infinite. For this type of medium, time-of-flight methods are used to estimate the shear wave velocity.

For a viscoelastic medium, the wave velocity varies with frequency, a phenomenon called dispersion. Multiple SWE methods have taken advantage of this phenomenon by measuring the shear wave phase velocity at different frequencies to evaluate the viscoelasticity of a medium. In addition to viscoelasticity, geometry of a medium can also cause dispersion. Waves propagating in materials that are thin, with respect to the wavelength of the wave, can undergo multiple reflections resulting in complex wave behavior with multiple modes and variation of phase velocity with frequency. There are also situations where tissues have finite thicknesses and are viscoelastic, such as arteries, myocardium, bladder wall, and tendons.

The dispersion of shear waves in tissue-mimicking materials has been measured to characterize the viscoelastic mechanical properties. In some previous methods, a phase gradient was used to calculate the phase velocity. The phase gradient at a frequency, f, is calculated as,

$${c}_{s} ( ω ) = \frac{ωΔ x}{Δφ} ; \qquad ( 1 )$$

where ω=2πf, Δx=x₂−x₁, and Δϕ=ϕ₂−ϕ₁. The shear wave phases, ϕ₂ and ϕ₁, measured at frequency, f, are measured at two locations x₁ and x₂. The phase can be calculated by taking a Fourier transform of the motion at a given location and extracting the phase of that signal at the frequency of interest. This operation can be repeated for all locations in the data set. Commonly, this phase gradient is measured at several locations, and the slope of the line m=Δx/Δϕ, which can be measured using linear regression or curve fitting, is used in Eqn. (1) to calculate the phase velocity.

In most current applications, the shear wave propagation is measured at multiple lateral locations using ultrafast imaging techniques either using plane wave compounding or by using multiple acquisitions with focused beams. In many cases, the particle velocity, v, is used for data processing, but displacement or acceleration could also be used. The two-dimensional Fourier transform (2D-FT) of this spatiotemporal particle motion, v(x,t), is used to create the “k-space” represented by V(k,f), where k is the spatial frequency and f is the temporal frequency. The peaks of this k-space represents the phase velocities of different wave propagation modes. These peaks can be found by searching orthogonal directions, either along the f-direction or k-direction. Typically, a threshold is applied to the k-space before the search to avoid spurious peaks. The coordinates of the peaks are used to calculate the phase velocity as cₛ(f)=f/k.

Another way to search the k-space that has been proposed is a Radon sum method which uses the k-space and finds the curved trajectory defined by a linear dispersion function that maximizes the summed magnitude. Any dispersion function could be defined for this method, but the parameters that provide the maximized sum are reported for characterization of the medium under investigation.

For accurate and robust characterization of viscoelastic media, algorithms to reliably extract the phase velocity dispersion are desired. Phase gradient and 2D-FT methods can, at times, be prone to failure in the face of experimental noise. Therefore, methods that provide stable dispersion curve estimates are still desired.

### SUMMARY OF THE DISCLOSURE

The present disclosure addresses the aforementioned drawbacks by providing a method for estimating a phase velocity of a shear wave using an ultrasound system. Ultrasound data acquired with an ultrasound system from a region-of-interest in a subject in which a shear wave was propagating when the ultrasound data were acquired are provided to a computer system. The ultrasound data comprise at least one spatial dimension and at least one temporal dimension. Temporal frequency data are generated by Fourier transforming the ultrasound data along the at least one temporal dimension. For each temporal frequency in the temporal frequency data, an autocorrelation matrix is computed from the temporal frequency data associated with the temporal frequency. Eigenvecotrs in each autocorrelation matrix are separated into a shear wave signal eigenvector group and a noise signal eigenvector group based on a sorting of eigenvalues in each autocorrelation matrix. A wavenumber spectrum is computed based at least in part on the eigenvectors in the noise signal eigenvector group. A phase velocity of the shear wave is then estimated based at least in part on the wavenumber spectrum.

The foregoing and other aspects and advantages of the present disclosure will appear from the following description. In the description, reference is made to the accompanying drawings that form a part hereof, and in which there is shown by way of illustration a preferred embodiment. This embodiment does not necessarily represent the full scope of the invention, however, and reference is therefore made to the claims and herein for interpreting the scope of the invention.

### BRIEF DESCRIPTION OF THE DRAWINGS

FIG. 1 is a flowchart setting forth the steps of an example method for estimating a phase velocity of a shear wave using an ultrasound system.

FIG. 2 is an example of a shear wave signal eigenvector subspace produced using the methods described in the present disclosure.

FIG. 3 is an example of a noise signal eigenvector subspace produced using the methods described in the present disclosure.

FIG. 4 is an example of a wavenumber spectrum produced using the methods described in the present disclosure.

FIGS. 5A-5C show cross-sections for a single temporal frequency of a wavenumber spectrum produced using the methods described in the present disclosure. FIG. 5A corresponds to a wavenumber spectrum based on ultrasound data measured at a depth of 30 mm, FIG. 5B corresponds to a wavenumber spectrum based on ultrasound data measured at a depth of 45 mm, and corresponds to a wavenumber spectrum based on ultrasound data measured at a depth of 70 mm.

FIG. 6 shows an example of time-domains shear wave signals measured at various focal depths.

FIG. 7 is a block diagram of an example ultrasound system that can implement the methods described in the present disclosure.

FIG. 8 is a block diagram of an example computer system that can implement the methods described in the present disclosure.

FIGS. 9A-9I show example experimental data results based on the methods described in the present disclosure. The top row presents spatiotemporal particle velocity. The k-space spectra calculated using 2D-FT (middle row) and MUSIC (bottom row) methods. Results were calculated for the numerical FEM viscoelastic phantoms without added noise, with assumed material properties: (a), (d), (g) G₀=10 kPa, G_{∞}=2 kPa, β=6667 1/s (Phantom 1); (b), (e), (h) G₀=15 kPa, G_{∞}=4 kPa, β=5500 1/s (Phantom 2); (c), (f), (i) G₀=20 kPa, G_{∞}=4 kPa, β=4000 1/s (Phantom 3). Dash-dotted lines correspond to the polynomial fitting results.

FIGS. 10A-10I show example experimental data results based on the methods described in the present disclosure. The top row presents spatiotemporal particle velocity. The k-space spectra calculated using 2D-FT (middle row) and MUSIC (bottom row) methods. Results were calculated for the numerical FEM viscoelastic phantoms with an SNR of 20 dB, with assumed material properties: (a), (d), (g) G₀=10 kPa, G_{∞}=2 kPa, β=6667 1/s (Phantom 1); (b), (e), (h) G₀=15 kPa, G_{∞}=4 kPa, β=5500 1/s (Phantom 2); (c), (f), (i) G₀=20 kPa, G_{∞}=4 kPa, β=4000 1/s (Phantom 3). Dash-dotted lines correspond to the polynomial fitting results.

FIGS. 11A-11F show example experimental data results based on the methods described in the present disclosure. Comparison of phase velocities calculated based on various approaches. Results were estimated for the numerical FEM viscoelastic phantoms without (top row) and with (bottom row) manually added white Gaussian noise, with assumed material properties: (a), (d), G₀=10 kPa, G_{∞}=2 kPa, β=6667 1/s (Phantom 1); (b), (e), G₀=15 kPa, G_{∞}=4 kPa, β=5500 1/s (Phantom 2); (c), (f), G₀=20 kPa, G_{∞}=4 kPa, β=4000 1/s (Phantom 3).

FIGS. 12A-12I show example experimental data results based on the methods described in the present disclosure. The RMSE calculated in a frequency range from 250 to 1400 Hz at a radiation force focused, at depth of 30 mm (top row), 50 mm (middle row) and 70 mm (bottom row) for the numerical FEM data with assumed material properties of: (a), (d), (g) G₀=10 kPa, G_{∞}=2 kPa, β=6667 1/s (Phantom 1); (b), (e), (h) G₀=15 kPa, G_{∞}=4 kPa, β=5500 1/s (Phantom 2); (c), (f), (i) G₀=20 kPa, G_{∞}=4 kPa, β=4000 1/s (Phantom 3).

FIGS. 13A-13C show example experimental data results based on the methods described in the present disclosure. Phase velocities calculated using various approaches (discussed in this section) for a threshold value of (a) 0.05, (b) 0.1 and (c) 0.15. Data were processed for a radiation force focused, at depth of 50 mm, for the numerical FEM data with assumed material properties of G₀=15 kPa, G_{∞}=4 kPa, and β=5500 1/s (Phantom 2). SNR value was set to 20 dB.

FIGS. 14A-14I show example experimental data results based on the methods described in the present disclosure. The k-space spectra calculated using the 2D-FT (top row) and MUSIC (middle row) methods, and comparison of phase velocities calculated based on various approaches (bottom row). Results were calculated for the QIBA phantom type A with a focal depth of: (a), (d), (g) 30 mm, (b), (e), (h) 45 mm and (c), (f), (i) 70 mm. Dash-dotted lines in the k-space spectra correspond to a polynomial fitting.

FIGS. 15A-15I show example experimental data results based on the methods described in the present disclosure. The k-space spectra calculated using the 2D-FT (top row) and MUSIC (middle row) methods, and comparison of phase velocities calculated based on various approaches (bottom row). Results were calculated for the QIBA phantom type B with a focal depth of: (a), (d), (g) 30 mm, (b), (e), (h) 45 mm and (c), (f), (i) 70 mm. Dash-dotted lines in the k-space spectra correspond to a polynomial fitting.

FIGS. 16A-16I show example experimental data results based on the methods described in the present disclosure. The k-space spectra calculated using the 2D-FT (top row) and MUSIC (middle row) methods, and comparison of phase velocities calculated based on various approaches (bottom row). Results were calculated for the QIBA phantom type C with a focal depth of: (a), (d), (g) 30 mm, (b), (e), (h) 45 mm and (c), (f), (i) 70 mm. Dash-dotted lines in the k-space spectra correspond to a polynomial fitting.

### DETAILED DESCRIPTION

Described here are methods for estimating the phase velocity of a shear wave from ultrasound data. An ultrasound system is used to measure one or more shear waves propagating in a region-of-interest in a subject, and from this data the phase velocity of the one or more shear waves can be estimated. In general, the phase velocity is estimated from a wavenumber spectrum computed from temporal frequency data generated by Fourier transforming the ultrasound data along one or more temporal dimensions. A multiple signal classification (i.e., “MUSIC”) technique is adapted to separate the temporal frequency data into signal and noise subspaces, from which wavenumber spectra can be estimated based, at least in part, on an estimation function.

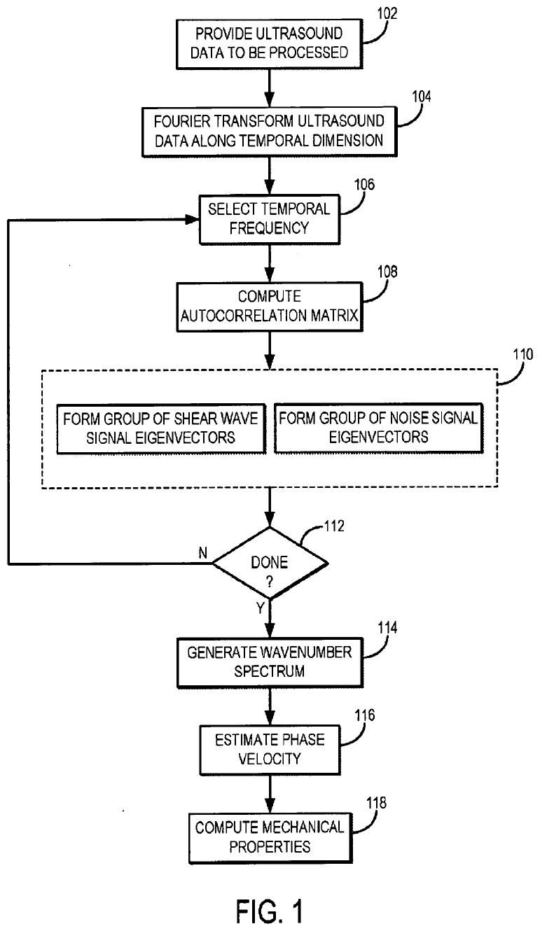

Referring now to FIG. 1, a flowchart is illustrated as setting forth the steps of an example method for estimating a shear wave phase velocity using a multiple signal classification technique. The method includes providing ultrasound data to a computer system for processing, as indicated at step 102. Providing the ultrasound data can include retrieving previously acquired ultrasound data from a memory or other data storage, or can include acquiring the ultrasound data with an ultrasound system and communicating the ultrasound data to the computer system, which may be a part of the ultrasound system.

In either case, the ultrasound data are acquired from a region-of-interest through which one or more shear waves are propagating. For instance, the ultrasound data can be acquired using a pulse sequence in which one or more push pulses are applied to the subject to induce shear waves in the region-of-interest, and in which one or more detection beams are applied to the subject to acquire the ultrasound data while the shear waves are propagating in the region-of-interest. The ultrasound data generally include one or more spatial dimensions and one or more temporal dimensions. The ultrasound data thus represent a number of different time frames that depict the region-of-interest while the shear waves were propagating in that region. In some implementations, the ultrasound data are acquired with uniform spatial sampling.

The ultrasound data are then Fourier transformed along the one or more temporal dimensions in order to generate temporal frequency data, as indicated at step 104. A temporal frequency associated with the temporal frequency data is selected, as indicated at step 106. For the selected temporal frequency, an autocorrelation matrix is computed from the spectrum associated with the selected frequency, as indicated at step 108.

The eigenvalues of each autocorrelation matrix are arranged to separate the related eigenvectors into two groups, as indicated at step 110. Each autocorrelation matrix, Rₓ, is an M×M matrix, where IM is generally smaller than the length of the input data vector. As one non-limiting example, M can be selected as one-eighth of the length of a vector used for padding of the wave motion vectors with trailing zeros. In general, the eigenvectors are separated into a group of p eigenvectors associated with shear wave signals, and a groups of M−p eigenvectors associated with noise. As one non-limiting example, p can be set as the number of expected complex exponentials in the ultrasound data. For instance, if only one complex exponential is expected, then p can be set to unity.

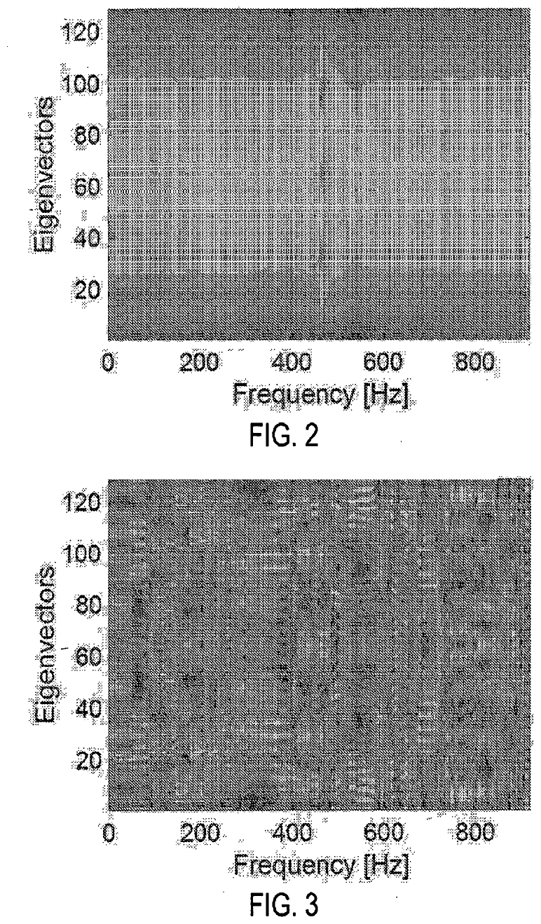

For instance, the eigenvalues in a given autocorrelation matrix can be arranged in decreasing order and the eigenvectors associated with the first p eigenvalues can be selected as the group of eigenvectors associated with the shear wave signals, while the eigenvectors associated with the next M−p eigenvalues can be selected as the group of eigenvectors associated with noise signals. An example of the p shear wave signal subspace associated with the shear wave signal eigenvectors is shown in FIG. 2, and an example of the noise subspace associated with the M−p noise eigenvectors is shown in FIG. 3. Differences between the shear wave signal eigenvectors (a smooth pattern) and the noise eigenvector (a random pattern) are noticeable. It is contemplated that the eigenvectors of the autocorrelation matrix, Rₓ, will have p roots that lie on a unit circle at the temporal frequencies of the complex exponential. Similarly, it is contemplated that the eigenspectrum associated with the noise eigenvectors will exhibit sharp peaks.

Referring again to FIG. 1, a decision is made a decision block 112 whether the desired temporal frequency data have been processed. If not, the next temporal frequency is selected at step 106 and an autocorrelation matrix is computed at step 108 and the eigenvectors separated into shear wave signal and noise signal groups at step 110.

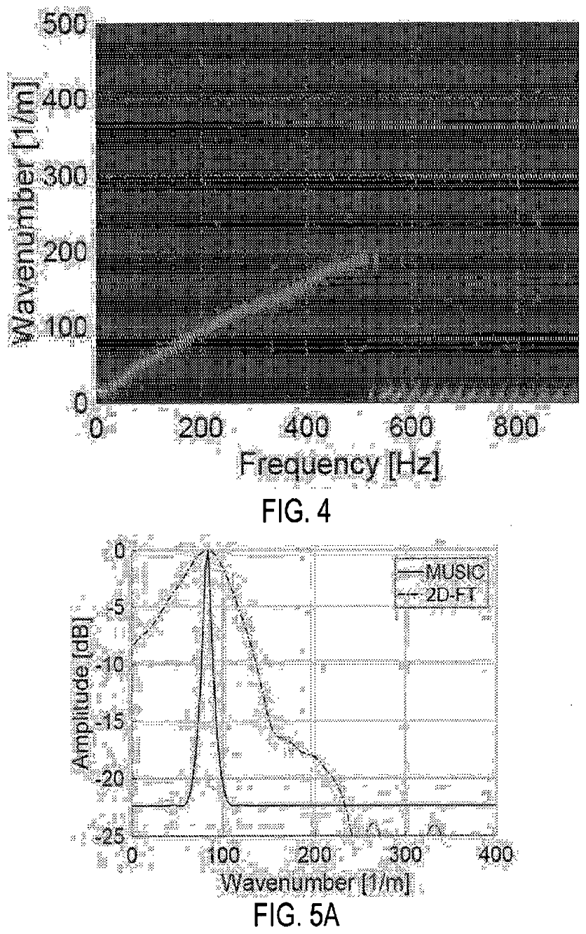

Using the M−p noise subspace eigenvectors, a wavenumber spectrum is computed, as indicated at step 114. For instance, the wavenumber spectrum, which can contain robust peaks that corresponds to the phase velocity of the major shear wave component, can be calculated using the following estimation function,

$$\hat{P} ( {e}^{jk} ) = {( {\sum }_{i = p + 1}^{M} {\vert {\overline{e}}^{H} {\overline{v}}_{i} \vert}^{2} )}^{- 1} ; \qquad ( 2 )$$

where vᵢ is the i^{th} eigenvector of the correlation matric, Rₓ, and ē^{H} is the vector of complex exponentials, e^{jkω}^{ₜ}. The superscript H denotes the Hermitian operator. The eigenvectors vᵢ used in the summation correspond to the M−p smallest eigenvalues that span the noise subspace. Based on Eqn. (2), the wavenumbers of the complex exponentials are taken as the locations of the p maximum peaks in \hat{P}(e^{jk}). An example of a wavenumber spectrum generated using Eqn. (2) and based on the eigenvector subspaces shown in FIGS. 2 and 3 is shown in FIG. 4. The visible change of eigenvectors for the shear wave signal subspace at a frequency of 530 Hz (FIG. 2) reflects the end of a shear wave mode on the wavenumber spectrum shown in FIG. 4.

Referring again to FIG. 1, using the wavenumber peaks, phase velocities are computed, as indicated at step 116. For instance, phase velocities can be computed as,

$$c = \frac{f}{k} ; \qquad ( 3 )$$

where f is the temporal frequency value and k is a wavenumber coordinate. From the phase velocities, mechanical properties can be computed for the region-of-interest, as indicated at step 118. The mechanical properties may then be stored for later use or presented to a user. As one example, the mechanical properties can be arranged in a digital image (e.g., a mechanical property map) that is displayed to a user in order to provide a visual depiction of the spatial distribution of a particular mechanical property in the region-of-interest. The mechanical properties could include, but are not limited to, stiffness, storage modulus, loss modulus, damping ratio, poroelastic parameters, viscoelastic parameters (e.g., viscoelastic moduli), Young's modulus, Poisson's ratio, shear modulus, bulk modulus, and so on.

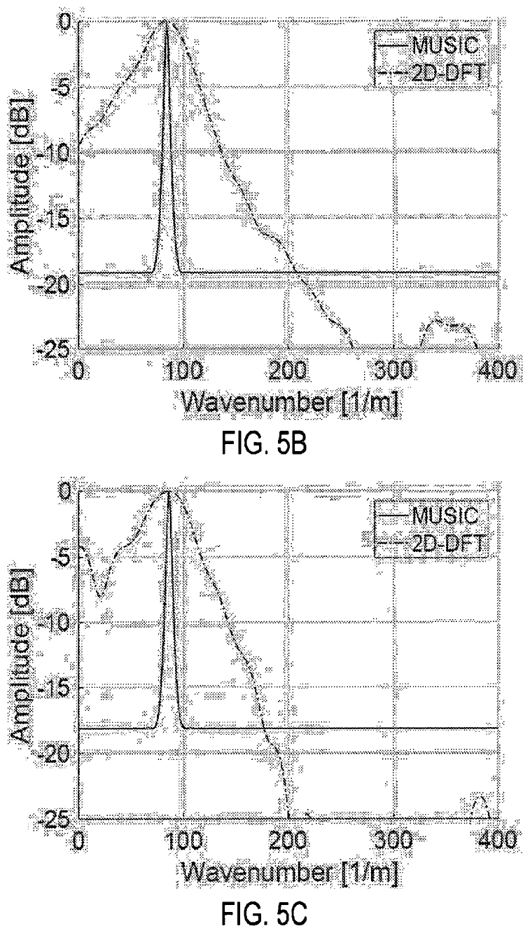

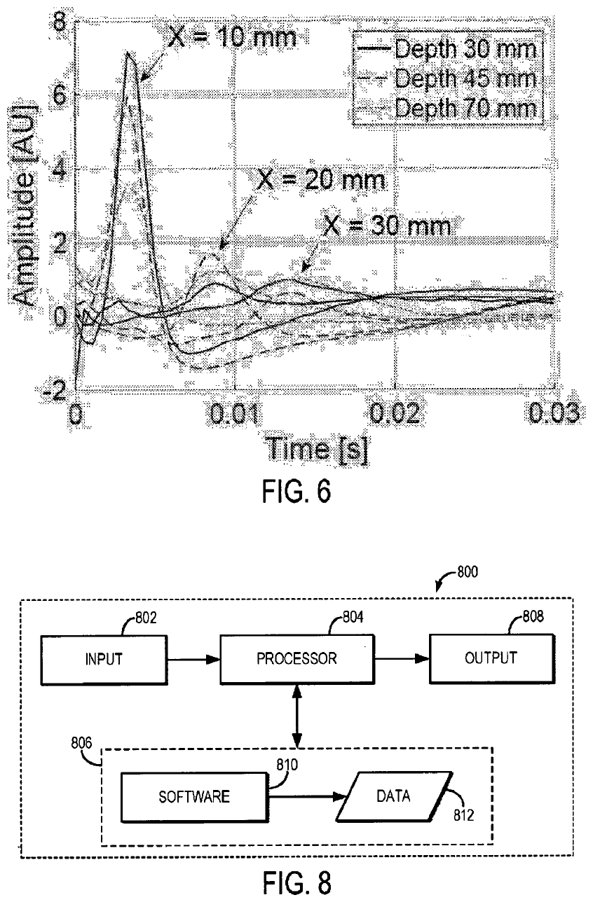

Comparisons of wavenumber spectra computed using the methods described in the present disclosure and a classical 2D-FT method are shown in FIGS. 5A-5C. These example computations were done for an acoustic radiation force focused and shear waves measured at depth of 30 mm (FIG. 5A), 45 mm (FIG. 5B), and 70 mm (FIG. 5C). The presented data are selected for a single temporal frequency of 180 Hz. The methods of the present disclosure produce wavenumber spectra exhibiting a sharp peak. Results gathered from the classical 2D-FT approach have a wider peak profile as well as side lobes caused by noise occurring in the input data, which result in ambiguities in phase velocity estimation. Similarly, the methods described in the present disclosure can produce a robust pseudospectrum estimate of the input shear wave signals, as shown in FIG. 6.

Different methods can be used to search the wavenumber-temporal frequency space generated by the methods described in the present disclosure. As one example, a peak detection method can be used to find the wavenumber values where there are local or global maxima at a given temporal frequency value. As another example, finding wavenumber peaks can be based on computing a gradient of the wavenumber space magnitude distribution and finding the associated zero-crossings that correspond to the peaks.

For a two-dimensional function, g(x; y), the gradient is given as,

$$∇ g = \frac{δ g}{δ x} i + \frac{δ g}{δ y} j . \qquad ( 4 )$$

Considering a one-dimensional function with a peak at x=xₚ, the derivative of that function will have a zero-crossing at x=xₚ. Detections of zero-crossings could be used instead of peaks in the magnitude distribution along a given search direction, either temporal frequency, f, or wavenumber, k. In some instances, a Fourier-based derivative can be performed using,

$$\frac{d}{dx} g ( x ) = Re \{ {FFT}^{- 1} [ {ik}_{x} FFT ( g ( x ) ) ] \} ; \qquad ( 5 )$$

where Re { . . . } indicates taking the real component, and kₓ is the Fourier-domain variable corresponding to x. This technique for taking the derivative is performed in both the temporal and spatial dimensions and the results are summed to compute the gradient of the wavenumber space.

Using the methods described in the present disclosure, the vectorial nature of the gradient does not need to be retained; rather, the following implementation can be used,

$$∇ f = \frac{δ g}{δ x} + \frac{δ g}{δ y} . \qquad ( 6 )$$

Phase velocities estimated based on the methods described in the present disclosure were studied based on both the maximum peaks and the gradient techniques described above. The phase velocities were also studied for fitted wavenumber-frequency pairs for the gradient method only. In this latter approach, a ninth-order polynomial curve fitting was adopted for the wavenumber space peak or zero-crossing locations. The order of the polynomial can be changed to an approximate value as the application dictates, or as otherwise desired by the user. Next, for the fitted wavenumber-frequency curves, phase velocities were calculated and compared with non-fitted results.

The calculation of the wave velocities, c=f/k, involves a division of the values of the wavenumber coordinates; thus, if the data exhibit significant spread, the wave velocities may also have a large variation. Applying a least-squares polynomial fitting can reduce that variation.

Thus, methods for estimating wavenumber spectra and phase velocities for shear waves from ultrasound data have been described. In general, one-dimensional time-domain Fourier transform is implemented and a multiple signal classification approach is adapted to find the wavenumber-frequency content of the shear wave signal. In addition to a maximum peak method for identifying the dispersion curves in wavenumber space, a gradient-based method can be used to find zero-crossings that represent the phase velocities. In some other implementations, a polynomial to fit wavenumber data points identified with the gradient-based search method can be used.

The methods described in the present disclosure do not require tuning parameters to be used; rather, there is one optimal value that provides the most accurate measurements. As one non-limiting example, the size of the autocorrelation matrix, M, can be tuned (e.g., treated as a noise subspace eigenfilter), as well as the p value representing the number of main components present in a the ultrasound data signal.

For the curve fitting described above, a ninth-order polynomial was used. It is contemplated that in some cases the polynomial will sharply change when peaks are difficult to be identified. The choice of the ninth-order polynomial as the curve fitting model can be used to make the curve smooth while still being able to adapt to the data adequately. In other implementations, a polynomial different than a ninth-order polynomial can also be used.

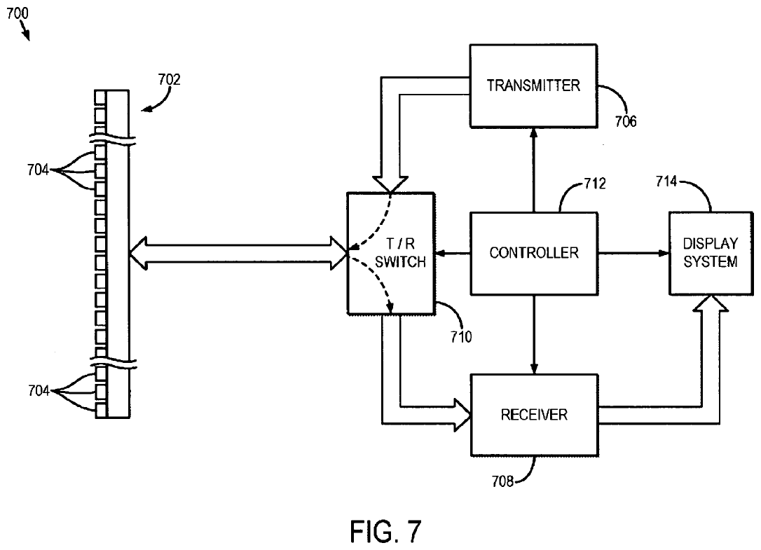

FIG. 7 illustrates an example of an ultrasound system 700 that can implement the methods described in the present disclosure. The ultrasound system 700 includes a transducer array 702 that includes a plurality of separately driven transducer elements 704. The transducer array 702 can include any suitable ultrasound transducer array, including linear arrays, curved arrays, phased arrays, and so on.

When energized by a transmitter 706, each transducer element 702 produces a burst of ultrasonic energy. The ultrasonic energy reflected back to the transducer array 702 from the object or subject under study (e.g., an echo) is converted to an electrical signal (e.g., an echo signal) by each transducer element 704 and can be applied separately to a receiver 708 through a set of switches 710. The transmitter 706, receiver 708, and switches 710 are operated under the control of a controller 712, which may include one or more processors. As one example, the controller 712 can include a computer system.

The transmitter 706 can transmit unfocused or focused ultrasound waves. In some configurations, the transmitter 706 can also be programmed to transmit diverged waves, spherical waves, cylindrical waves, plane waves, or combinations thereof. Furthermore, the transmitter 706 can be programmed to transmit spatially or temporally encoded pulses.

The receiver 708 can be programmed to implement a suitable detection sequence for the imaging task at hand. In some embodiments, the detection sequence can include one or more of line-by-line scanning, compounding plane wave imaging, synthetic aperture imaging, and compounding diverging beam imaging.

Thus, in some configurations, the transmitter 706 and the receiver 708 can be programmed to implement a high frame rate. For instance, a frame rate associated with an acquisition pulse repetition frequency (“PRF”) of at least 100 Hz can be implemented. In some configurations, the ultrasound system 700 can sample and store at least one hundred ensembles of echo signals in the temporal direction.

The controller 712 can be programmed to design an imaging sequence using the techniques described in the present disclosure, or as otherwise known in the art. In some embodiments, the controller 712 receives user inputs defining various factors used in the design of the imaging sequence, which may include parameters associated with inducing shear waves in a region-of-interest, parameters associated with detecting shear wave signals, and so on.

A scan can be performed by setting the switches 710 to their transmit position, thereby directing the transmitter 706 to be turned on momentarily to energize each transducer element 704 during a single transmission event according to the designed imaging sequence. The switches 710 can then be set to their receive position and the subsequent echo signals produced by each transducer element 704 in response to one or more detected echoes are measured and applied to the receiver 708. The separate echo signals from each transducer element 704 can be combined in the receiver 708 to produce a single echo signal. Images produced from the echo signals can be displayed on a display system 714.

In some embodiments, the receiver 708 may include a processing unit, which may be implemented by a hardware processor and memory, to process echo signals or images generated from echo signals. As an example, such a processing unit can estimate phase velocities of shear waves using the methods described in the present disclosure.

Referring now to FIG. 8, a block diagram of an example of a computer system 800 that can perform the methods described in the present disclosure is shown. The computer system 800 generally includes an input 802, at least one hardware processor 804, a memory 806, and an output 808. Thus, the computer system 800 is generally implemented with a hardware processor 804 and a memory 806.

In some embodiments, the computer system 800 can be the processing unit of an ultrasound system, such as those described above. The computer system 800 may also be implemented, in some examples, by a workstation, a notebook computer, a tablet device, a mobile device, a multimedia device, a network server, a mainframe, one or more controllers, one or more microcontrollers, or any other general-purpose or application-specific computing device.

The computer system 800 may operate autonomously or semi-autonomously, or may read executable software instructions from the memory 806 or a computer-readable medium (e.g., a hard drive, a CD-ROM, flash memory), or may receive instructions via the input 802 from a user, or any another source logically connected to a computer or device, such as another networked computer or server. Thus, in some embodiments, the computer system 800 can also include any suitable device for reading computer-readable storage media.

In general, the computer system 800 is programmed or otherwise configured to implement the methods and algorithms described in the present disclosure. For instance, the computer system 800 can be programmed to estimate phase velocities of shear waves using the methods described in the present disclosure.

The input 802 may take any suitable shape or form, as desired, for operation of the computer system 800, including the ability for selecting, entering, or otherwise specifying parameters consistent with performing tasks, processing data, or operating the computer system 800. In some aspects, the input 802 may be configured to receive data, such as data acquired with an ultrasound system. Such data may be processed as described above to estimate phase velocities of shear waves propagating in a region-of-interest in a subject or other object being imaged. In addition, the input 802 may also be configured to receive any other data or information considered useful for estimating the phase velocities of shear waves using the methods described above.

Among the processing tasks for operating the computer system 800, the one or more hardware processors 804 may also be configured to carry out any number of post-processing steps on data received by way of the input 802. For instance, the one or more hardware processors 804 may also be configured to estimate mechanical properties based on the phase velocities computed using the methods described in the present disclosure.

The memory 806 may contain software 810 and data 812, such as data acquired with an ultrasound system, and may be configured for storage and retrieval of processed information, instructions, and data to be processed by the one or more hardware processors 804. In some aspects, the software 810 may contain instructions directed to estimating phase velocities of shear waves propagating in a region-of-interest in a subject or object being imaged.

In addition, the output 808 may take any shape or form, as desired, and may be configured for displaying shear wave signals, noise signals, wavenumber spectra, phase velocity dispersion data, mechanical property data or maps, and images obtained with an ultrasound system, in addition to other desired information.

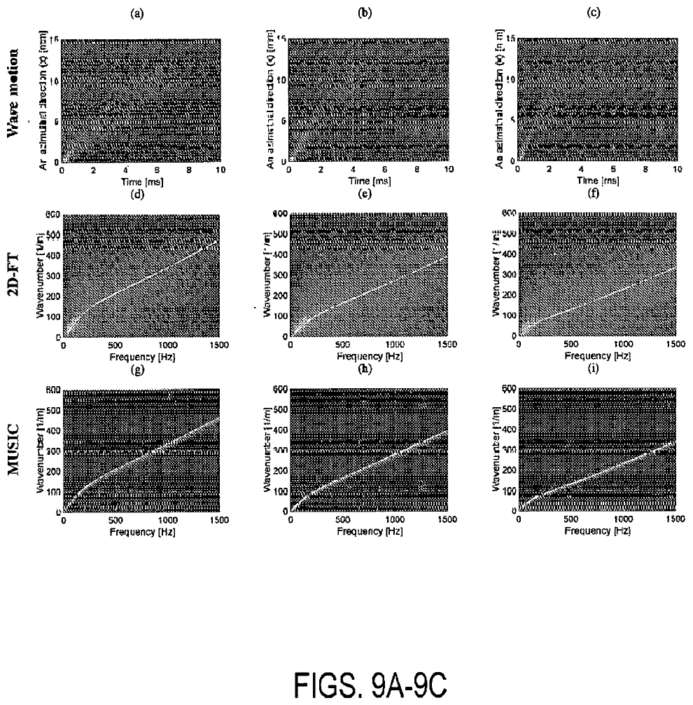

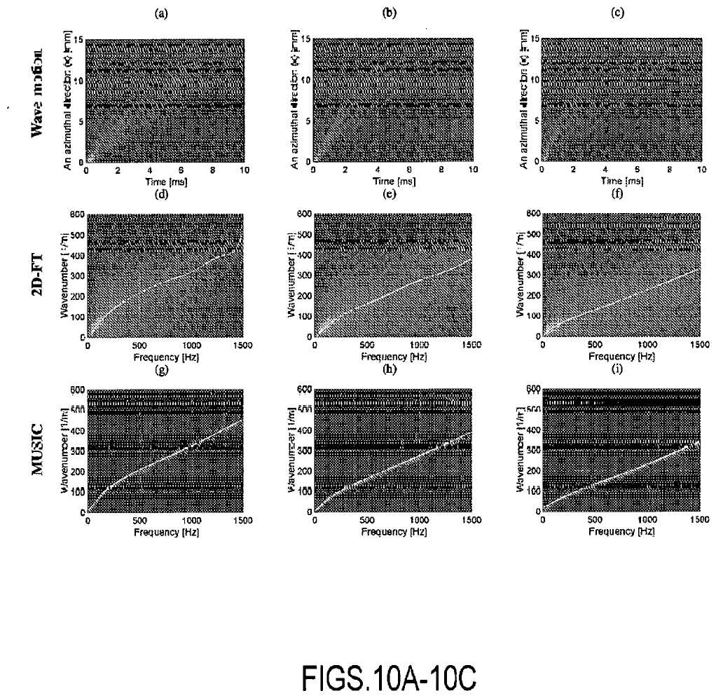

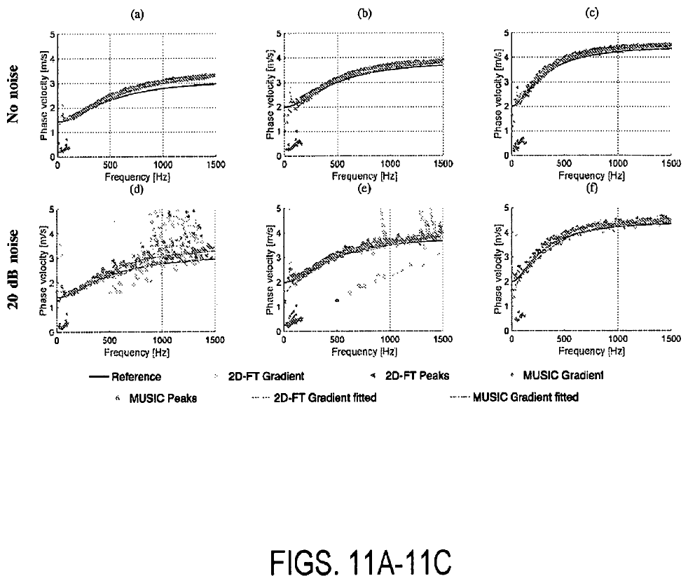

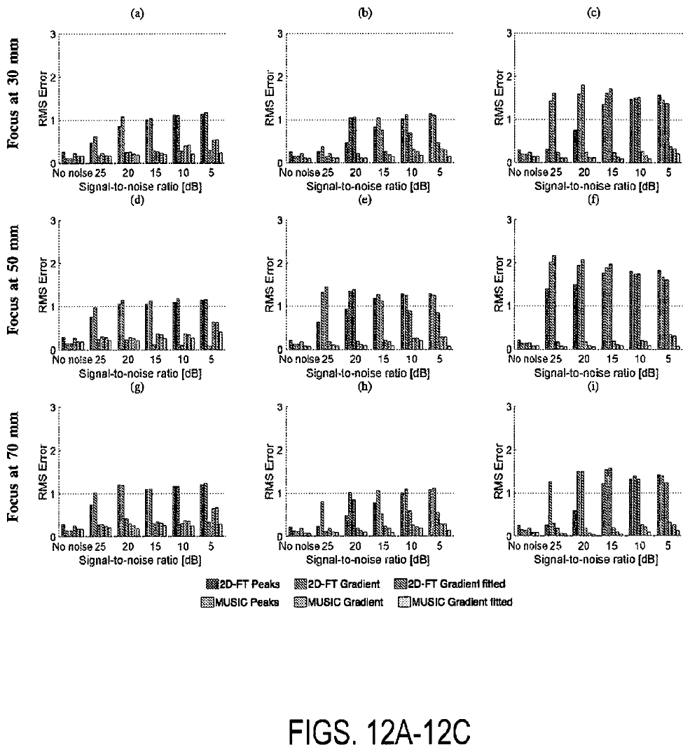

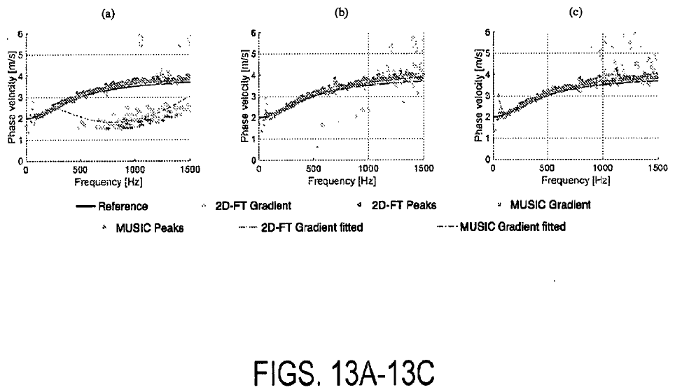

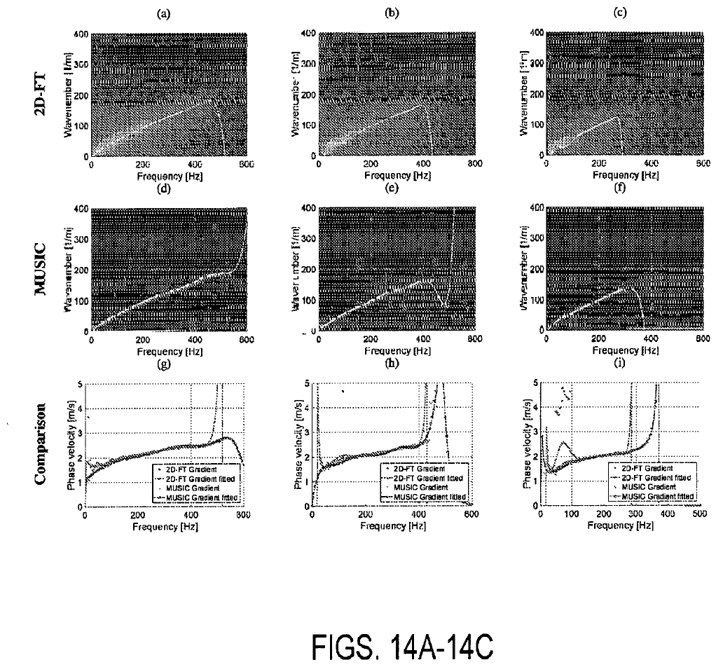

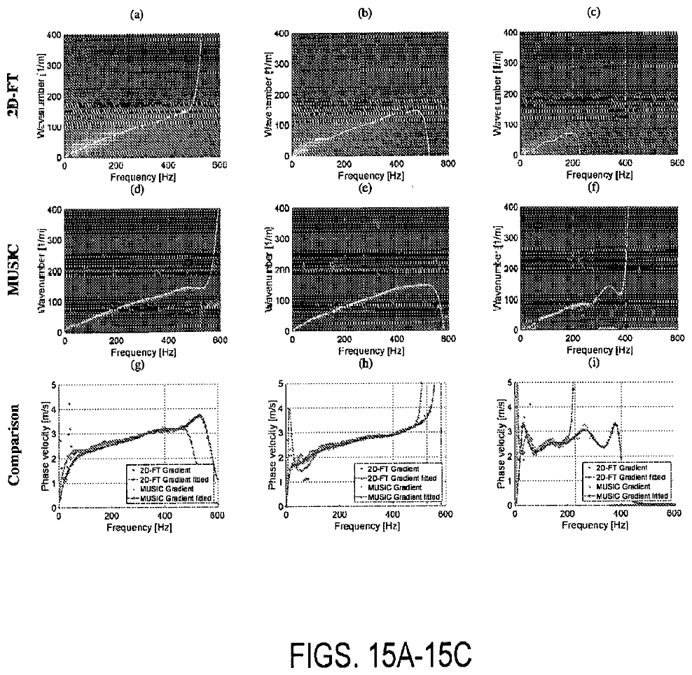

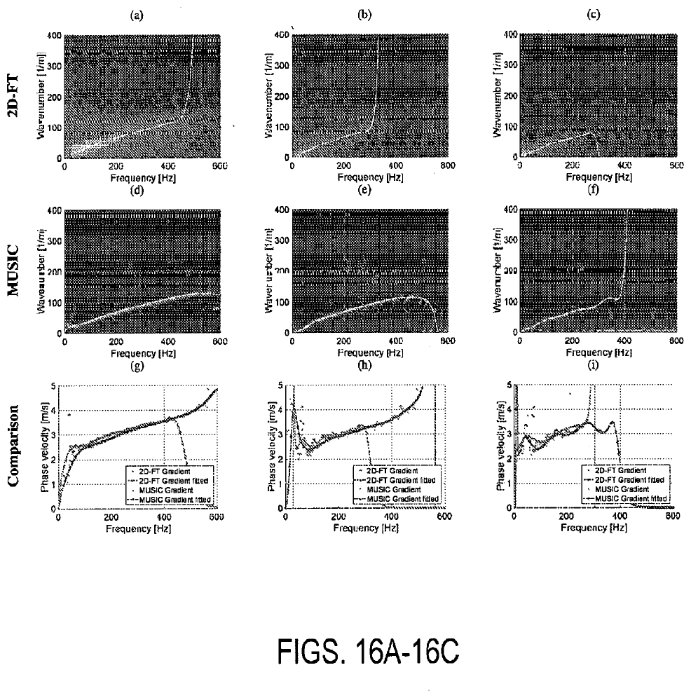

Examples of data acquired in test studies of the methods described in the present disclosure are shown in FIGS. 9-16. In general, these experimental data represent the methods described in the present disclosure being tested on data from finite element modeling simulations of shear wave propagation, and on data from viscoelastic elastography phantoms.

To produce digital phantoms of viscoelastic materials, for which the mechanical properties are known, finite element modeling was used. For these FEMs, Abaqus (6.12-1, Dassault Systems, Waltham, Mass.) was used. The acoustic radiation force push beam was simulated using Field II.

The array that was simulated was a curved linear array with a radius of curvature of 60 mm, element height of 14 mm, element pitch of 0.477 mm, elevation focus of 50 mm, center frequency of 3.0 MHz, and using a medium attenuation, α, of 0.45 dB/cm/MHz and sound speed, c, of 1540 m/s. The pressure was calculated and the intensity, I, was calculated by squaring the pressure to be used in the body force defined by F=2αI/c. Focal depths of 30, 50, and 70 mm were used for the push beams with a fixed F-number (F/N) of 2.

The viscoelastic domains were uniformly spatially sampled at 0.167 mm. The dimensions of the simulated domain were x=±25 mm in the azimuthal dimension, y=±10 mm in the elevation direction, and z=0-100 mm in the axial dimension. Simulations were performed with quarter-symmetry so the domain described by x=0-25 mm, y=0-10 mm, and z=0-100 mm was used for the calculations of motion. The viscoelasticity was modeled using Generalized Maxwell model.

The relaxation shear modulus Gᵣ of this model is,

Gᵣ(t)=G_{∞}+G₁e^{-βt} (7);

where G_{∞} is the long-term modulus, G₁ is the spring elasticity, and β is the decay constant of the relaxation modulus. The instantaneous shear modulus is,

G₀=G_{∞}+G₁ (8).

The decay constant is,

$$β = \frac{{G}_{1}}{{η}_{1}} ; \qquad ( 9 )$$

where η₁ is the damper viscosity.

Three different viscoelastic media of material properties tabulated in Table 1 were studied in this work.

TABLE 1
Numerical FEM Viscoelastic Phantom Parameters

| | G₀ [kPa] | G_{∞} [kPa] | β [1/s] |
|---|---|---|---|
| Phantom 1 | 10 | 2 | 6667 |
| Phantom 2 | 15 | 4 | 5500 |
| Phantom 3 | 20 | 4 | 4000 |

Numerical FEM shear wave responses for digital phantoms of viscoelastic materials were studied twofold. Examples of “clean” wave motions (without any additional noise) as well as wave motions in the presence of a noise (as added white Gaussian noise) were examined. The white Gaussian noise was generated and then added into shear wave time-domain signals. First, the power of the wave motion before adding noise was measured. Subsequently, white Gaussian noise was added to the time-domain vector signal. The signal-to-noise ratio (“SNR”) for the noise-added models was set to vary between 5-25 dB.

Three viscoelastic phantoms (CIRS, Inc., Norfolk, Va.) were used in a multi-center study conducted by the Radiological Society of North America Quantitative Imaging Biomarkers Alliance (RSNA QIBA) committee for standardizing shear wave speed measurement for the purposes of determining stage of liver fibrosis. These phantoms were designed such that each one represented a different stage of liver fibrosis. They are denoted phantoms A, B, and C.

Shear wave acquisitions were performed with a Verasonics system (Vantage, Verasonics, Inc., Kirkland, Wash.) and a curved linear array transducer (C5-2v, Verasonics, Inc., Kirkland, Wash.). Data were taken with different focal depths to explore biases related to acquisition depth. Acoustic radiation force push beams were focused at 30, 45, and 70 mm with an F-number of 2.2. The push duration was 800 is and the push frequency was 2.0 MHz. A wide beam acquisition using three beams that were coherently compounded was implemented. The effective frame rate after compounding was 1.8315 kHz. The motion (i.e., shear wave particle velocity) was calculated from the in-phase/quadrature data using an autocorrelation algorithm.

The present disclosure has described one or more preferred embodiments, and it should be appreciated that many equivalents, alternatives, variations, and modifications, aside from those expressly stated, are possible and within the scope of the invention.

1. A method for estimating a phase velocity of a shear wave using an ultrasound system, the steps of the method comprising:
(a) providing to a computer system, ultrasound data acquired with an ultrasound system from a region-of-interest in a subject in which a shear wave was propagating when the ultrasound data were acquired, wherein the ultrasound data comprise at least one spatial dimension and at least one temporal dimension;
(b) generating temporal frequency data by Fourier transforming the ultrasound data along the at least one temporal dimension;
(c) for each temporal frequency in the temporal frequency data, computing an autocorrelation matrix from the temporal frequency data associated with the temporal frequency;
(d) separating eigenvectors in each autocorrelation matrix into a shear wave signal eigenvector group associated with shear wave signals and a noise signal eigenvector group associated with noise based on a sorting of eigenvalues in each autocorrelation matrix;
(e) computing a wavenumber spectrum based at least in part on the eigenvectors in the noise signal eigenvector group; and
(f) estimating a phase velocity of the shear wave based at least in part on the wavenumber spectrum.

2. The method as recited in claim 1, further comprising computing mechanical properties of the region-of-interest based at least in part on the estimated phase velocity of the shear wave.

3. The method as recited in claim 1, wherein step (e) includes inputting the eigenvectors in the noise signal eigenvector group to an estimation function.

4. The method as recited in claim 3, wherein the estimation function includes multiplying the eigenvectors in the noise signal eigenvector group by a vector of complex exponentials associated with motion of the shear wave.

5. The method as recited in claim 4, wherein the vector of complex exponentials associated with motion of the shear wave is selected based on a priori knowledge of the shear wave propagating in the region-of-interest.

6. The method as recited in claim 1, wherein the phase velocity is estimated based on finding a peak in the wavenumber spectrum and computing the phase velocity based on wavenumber-temporal frequency data associated with the peak.

7. The method as recited in claim 6, wherein the peak in the wavenumber spectrum is found using a peak detection algorithm.

8. The method as recited in claim 6, wherein the peak in the wavenumber spectrum is found by computing a gradient of a wavenumber space magnitude distribution and finding a zero crossing in the gradient that corresponds to the peak.

9. The method as recited in claim 6, wherein the peak in the wavenumber spectrum is found by fitting wavenumber spectrum data to a polynomial using a curve fitting algorithm implemented with a hardware processor and a memory of the computer system.

10. The method as recited in claim 9, wherein the polynomial is a ninth-order polynomial.

11. The method as recited in claim 9, wherein an order of the polynomial is selected as an approximate value.

12. The method as recited in claim 1, further comprising generating a phase velocity map that depicts a spatial distribution of estimated phase velocity values in the region-of-interest in the subject.

13. The method as recited in claim 1, further comprising computing a mechanical property based on the estimated phase velocity.

14. The method as recited in claim 13, further comprising generating a mechanical property map that depicts a spatial distribution of estimated mechanical property values in the region-of-interest in the subject.

15. The method as recited in claim 13, wherein the mechanical property includes at least one of stiffness, storage modulus, loss modulus, damping ratio, poroelastic parameters, viscoelastic parameters, Young's modulus, Poisson's ratio, shear modulus, or bulk modulus.

16. The method as recited in claim 13, wherein the mechanical property comprises viscoelastic moduli computed from the estimated phase velocity.

17. The method as recited in claim 1, wherein each autocorrelation matrix is an M×M matrix and step (d) includes separating the eigenvectors in each autocorrelation matrix as p eigenvectors in the shear wave signal eigenvector group and M−p eigenvectors in the noise signal eigenvector group, wherein p is a selected number.

18. The method as recited in claim 16, wherein p is selected as a number of roots of the eigenvectors in the autocorrelation matrix that lie on a unit circle at temporal frequencies associated with complex exponentials corresponding to the ultrasound data.
